(19) United States
(12) Patent Application Publication
(10) Pub. No.: US 2026/0232202 A1
(43) Pub. Date: Aug. 13, 2026

(54) QUICK SCREENING METHOD FOR ASSESSING DIABETIC FOOT SYNDROME

(71) Applicants: Chi-Sheng HSIEH, Zhubei City (TW); Szu-Yuan CHEN, Taipei City (TW)

(72) Inventors: Chi-Sheng HSIEH, Zhubei City (TW); Szu-Yuan CHEN, Taipei City (TW)

(21) Appl. No.: 19/063,117

(22) Filed: Feb. 25, 2025

(30) Foreign Application Priority Data
Mar. 1, 2024 (TW) 113107489

(51) Int. Cl. A61B 5/01; A61B 5/00; G01J 5/00; G01J 5/04; G01J 5/08; G01J 5/48; G06T 7/00; H04N 23/21; H04N 23/23; H04N 23/45

(52) U.S. Cl. CPC A61B 5/015; A61B 5/0077; G01J 5/0025; G01J 5/04; G01J 5/0859; G01J 5/485; G06T 7/0016; H04N 23/21; H04N 23/23; H04N 23/45; G01J 2005/0077; G06T 2207/10048

(57) ABSTRACT

A quick screening method is provided for assessing diabetic foot syndrome for a person. Both feet of the person are positioned onto a testing plate that blocks visible light and permits passage of near infrared (NIR). A multi-spectral camera device is used to simultaneously capture a thermal image and an NIR image of soles of the feet from a side of the testing plate opposite to the feet. The thermal image is aligned with and overlaid onto the NIR image, thereby obtaining an assessment image of the feet. Based on the assessment image, a determination is made regarding whether a temperature difference between a predetermined portion of one foot and a corresponding portion of the other foot exceeds 2.2° C. A potential case of diabetic foot syndrome is reported when the temperature difference exceeds 2.2° C.

### CROSS-REFERENCE TO RELATED APPLICATION

This application claims priority to Taiwanese Invention Patent Application No. 113107489, filed on Mar. 1, 2024, the entire disclosure of which is incorporated by reference herein.

### FIELD

The disclosure relates to a quick screening method, and more particularly to a quick screening method for assessing diabetic foot syndrome.

### BACKGROUND

Diabetes is a chronic metabolic disease characterized by abnormal blood glucose levels, posing serious threats to the health of the cardiovascular system, kidneys, and extremities of the body (such as feet). Diabetes often initially presents without symptoms, or with symptoms that are too subtle to be noticed.

Diabetic foot syndrome primarily occurs due to diabetes-induced peripheral neuropathy, which impairs sensory perception in the feet, making injuries more likely to go unnoticed. Early signs of this complication may include cracks, blisters, excessive formation of granulation tissue, redness, and/or localized temperature elevation.

Conventional examinations for diabetic foot syndrome include assessment of deformities at pressure points, foot blood pressure tests, checking for unnoticed ulcers or wounds due to sensory loss, evaluating insufficient blood circulation, and determining whether the foot lacks sensation. These assessments for foot complications require involvement of professional healthcare providers and typically take around 20 to 30 minutes, which is time-consuming and unsuitable for automation.

### SUMMARY

Therefore, one object of the disclosure is to provide a quick screening method that is not only applicable to hospitals and clinics for initial assessment of diabetic foot syndrome, but also suitable for use in home care settings to facilitate self-assessment of diabetic foot syndrome.

Another object of this disclosure is to provide a quick screening method for early detection of potential risks associated with diabetic foot, so as to enable high-risk individuals to seek timely medical consultation. This aims to reduce the impact of diabetic foot and alleviate the associated economic burden.

Yet another object of this disclosure is to provide a quick screening method that is non-invasive, non-contact, painless, rapid, and cost-effective for assessment of diabetic foot syndrome.

Still another object of this disclosure is to provide a quick screening method for early assessment and identification of diabetic foot, aiming to reduce consumption of medical resources, alleviate workload of healthcare professionals, and decrease frequency with which patients in rural areas need to travel to urban medical institutions.

In general, a temperature difference between the feet is less than 1° C. According to medical literature, a temperature difference exceeding 2.2° C. or 4° F. between the feet is considered abnormal, and this criterion can aid in identifying pathological conditions associated with diabetic foot syndrome. Therefore, the quick screening method provided in this disclosure employs a multi-spectral camera device that is able to capture thermal images to assist in the assessment of diabetic foot syndrome. This method can also be used to obtain extensive data on foot temperature distribution and foot deformities in both healthy individuals and diabetic patients, which can be used for future research.

According to the disclosure, the quick screening method for assessing diabetic foot syndrome for a person includes: positioning both feet of the person onto a testing plate that blocks visible light and permits passage of near infrared (NIR); using a multi-spectral camera device to simultaneously capture a thermal image and an NIR image of soles of the feet of the person from a side of the testing plate that is opposite to the feet of the person; performing image processing to align the thermal image with the NIR image and overlay the thermal image and the NIR image, thereby obtaining an assessment image of the feet; determining, based on the assessment image, whether a temperature difference between a predetermined portion of one of the feet and a corresponding portion of the other one of the feet exceeds 2.2° C.; and reporting a potential case of diabetic foot syndrome in response to determining that the temperature difference exceeds 2.2° C.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a schematic diagram illustrating a scenario of performing a quick screening method for assessing diabetic foot syndrome in accordance with some embodiments.

FIG. 2 is a block diagram illustrating a multi-spectral camera device used for the quick screening method in accordance with some embodiments.

FIG. 3 is a perspective view illustrating a near infrared light source used for the quick screening method in accordance with some embodiments.

FIGS. 4 and 5 are perspective views illustrating the use of a magnetic clip-on filter module in accordance with some embodiments.

FIG. 6 is a schematic diagram illustrating the magnetic clip-on filter module in accordance with some embodiments.

FIG. 7 is a schematic diagram illustrating a variation of the magnetic clip-on filter module in accordance with some embodiments.

FIG. 8 is a schematic diagram illustrating some predetermined corresponding portions of the feet that can be used for temperature comparison in accordance with some embodiments.

FIG. 9 is a schematic diagram illustrating a variation of the quick screening method in accordance with some embodiments.

FIG. 10 is a schematic diagram illustrating a footprint in an assessment image, which can be used to assess changes in an area and/or a shape of a foot over time.

FIG. 11 is a schematic diagram illustrating another scenario of performing the quick screening method for assessing diabetic foot syndrome in accordance with some embodiments.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

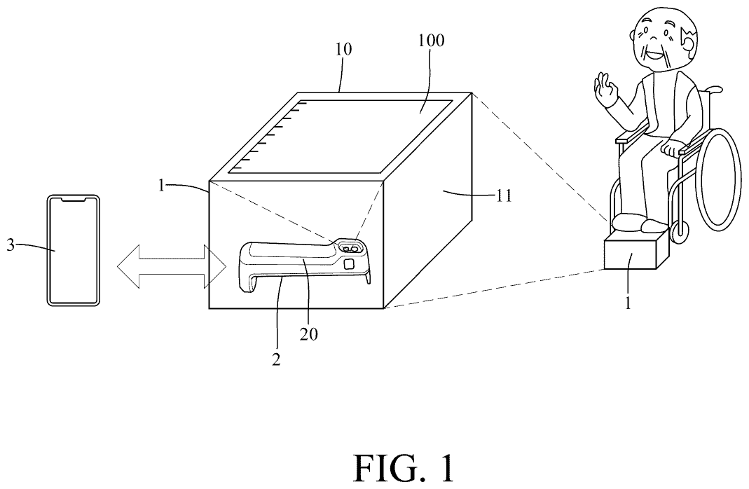

Referring to FIG. 1, in accordance with some embodiments, a quick screening method for assessing diabetic foot syndrome is implemented using a system that includes a testing box 1, a multi-spectral camera device 2, and a computerized device 3. The testing box 1 includes a main wall 10, and a continuous opaque wall 11 that is connected to a periphery of the main wall 10. The main wall 10 and the opaque wall 11 cooperatively define an accommodation space. The main wall 10 has a central portion formed by a testing plate 100 that is made of, for example, a glass material or a plastic material, and is configured to block visible light (VIS, typically having a wavelength range of from about 0.4 μm to about 0.8 μm) and permit passage of near infrared (NIR, typically having a wavelength range of from about 0.8 μm to about 1.0 μm). In accordance with some embodiments, the testing plate 100 may be made of, for example, a transparent resin (e.g., polymethylmethacrylate (PMMA), polycarbonate (PC), etc.) in which a black material is added. The black material may be, for example, a mixture of at least two of three primary color masterbatches (i.e., red masterbatch, green masterbatch and blue masterbatch) in accordance with some embodiments.

During the quick screening process, both feet of a person under testing are required to be positioned onto the testing plate 100. In FIG. 1, the multi-spectral camera device 2 is placed on the ground with its lens module facing upward, and is covered by the testing box 1, such that the multi-spectral camera device 2 is disposed in the accommodation space of the testing box 1, an inner surface of the testing plate 100 faces the lens module of the multi-spectral camera device 2, and an outer surface of the testing plate 100 faces upward. In the illustrative embodiment of FIG. 1, the feet are to be placed onto the outer surface of the testing plate 100 (so the feet and the multi-spectral camera device 2 are located at two opposite sides of the testing plate 100), and with the lens module of the multi-spectral camera device 2 facing the inner surface of the testing plate 100, the multi-spectral camera device 2 captures images of soles of the feet from an underside of the testing plate 100 (the side of the testing plate 100 that is opposite to the feet). In accordance with some embodiments, the testing plate 100 may be formed with some dimension markings, thereby aiding in estimation of an area and a shape of a footprint captured by the multi-spectral camera device 2 through the testing plate 100.

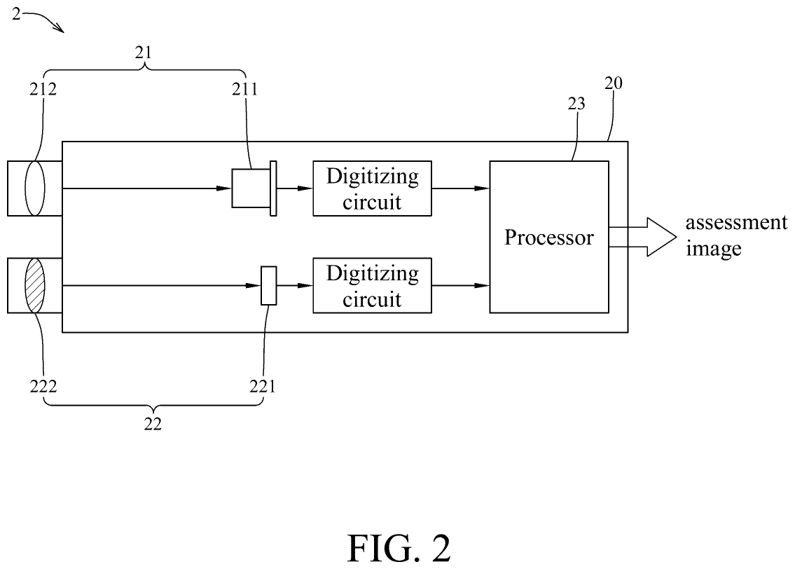

Further referring to FIG. 2, the multi-spectral camera device 2 includes a housing 20, a first camera module 21, a second camera module 22, and a processor 23. The first camera module 21 includes a first image sensor 211 accommodated in the housing 20, and a first lens 212 attached to a front surface of the housing 20 and corresponding in position to the first image sensor 211. The first image sensor 211 is sensitive to far infrared (FIR, typically having a wavelength range of from about 8 μm to about 14 μm) for capturing a thermal image in the direction of the soles of the feet, where the thermal image results from a temperature distribution on the testing plate 100, which corresponds to a temperature distribution of the soles of the feet. The second camera module 22 includes a second image sensor 221 accommodated in the housing 20, and a second lens 222 attached to the front surface of the housing 20 and corresponding in position to the second image sensor 221. The second image sensor 221 is sensitive to near infrared and visible light for capturing an NIR image and a VIS image in the direction of the soles of the feet.

The processor 23 is coupled to the first image sensor 211 and the second image sensor 221 for receiving the thermal image, the NIR image, and the VIS image therefrom, and is configured to perform image fusion by overlaying and aligning two or more of the thermal image, the NIR image, and the VIS image together, thereby obtaining an assessment image for assessing diabetic foot syndrome.

The computerized device 3 is communicatively connected to the multi-spectral camera device 2, and is configured to remotely control operation of the multi-spectral camera device 2, and to receive images from the multi-spectral camera device 2. In accordance with some embodiments, the computerized device 3 may be, for example, a smartphone, a tablet computer, a laptop computer, a desktop computer, etc., and is equipped with one or more software applications to enable interactions with the multi-spectral camera device 2.

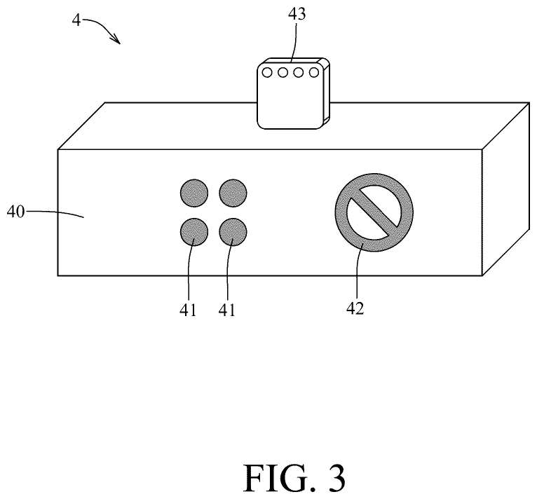

In general, NIR images exhibit higher contrast compared to VIS images and FIR images (i.e., thermal images), so NIR images can show more details or clearer contours of the feet than VIS images and FIR images can. In a case where visible light is too strong (e.g., stronger than near infrared) in a testing environment, an NIR light source may be used to illuminate the feet when the multi-spectral camera device 2 is capturing images of the feet, so as to strengthen the near infrared, and a clearer NIR image can be obtained. Referring to FIG. 3, an embodiment of an NIR light source 4 according to this disclosure is provided. The NIR light source 4 is configured to project near infrared toward the soles of the feet on the testing plate 100 (see FIG. 1), so as to strengthen the near infrared that is reflected by the feet. In accordance with some embodiments, the NIR light source 4 uses NIR light emitting diodes (LEDs) to emit near infrared. There are two common types of NIR LEDs on the market; one emits near infrared with a center wavelength of 850 nm, and the other emits near infrared with a center wavelength of 940 nm. In the illustrative embodiment, the NIR light source 4 includes a housing 40, an infrared source module having a plurality of NIR lighting components 41 that emit near infrared, a dimmer 42 operable to adjust intensity of the near infrared emitted by the NIR lighting components 41, and a connector 43 (e.g., a male micro/lightning USB connector) mounted to the housing 40. The connector 43 enables the NIR light source 4 to be connected to, for example, the multi-spectral camera device 2 or the computerized device 3 for acquiring electric power therefrom, but this disclosure is not limited in this respect. In accordance with some embodiments, the connector 43 may be omitted, and the NIR light source 4 may have its own power source, such as a battery (not shown) disposed within the housing 40. The infrared source module may be an infrared LED module having a plurality of NIR LEDs that serve as the NIR lighting components 41. The dimmer 42 may be realized using a variable resistor or a pulse width modulation (PWM) dimmer, and may include a knob for adjustment by a user. When the dimmer 42 is operated to increase an intensity of near infrared projected by the NIR light source 4, the NIR image of the feet would become clearer.

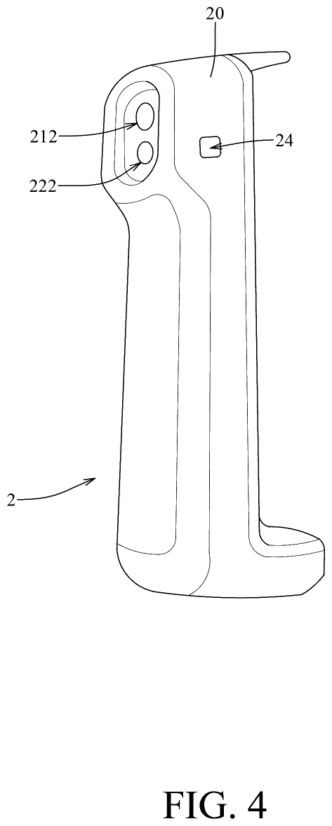

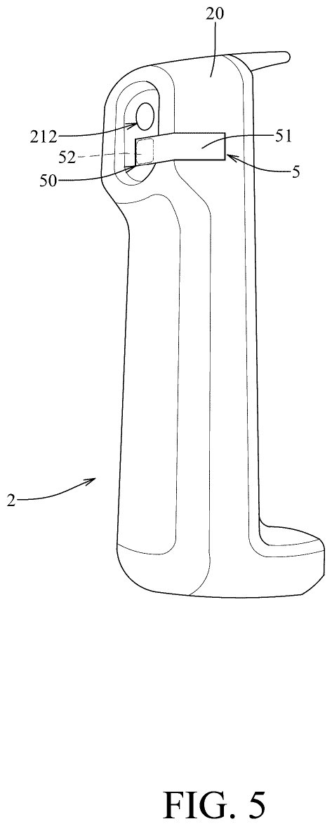

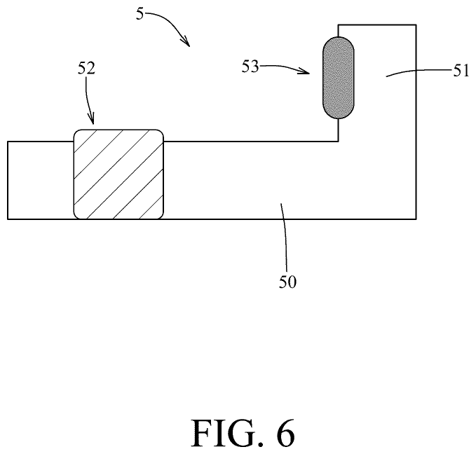

Referring to FIGS. 4 and 5, in accordance with some embodiments, the multi-spectral camera device 2 is adapted to be used with not only the NIR light source 4 (see FIG. 3), but also a magnetic clip-on filter module 5 that is configured to be detachably mounted to the housing 20 of the multi-spectral camera device 2. In this scenario, the multi-spectral camera device 2 has a metal piece 24 attached to a lateral surface of the housing 20 that is connected to the front surface of the housing 20 at an angle. Further referring to FIG. 6, the magnetic clip-on filter module 5 is L-shaped, and has a base plate 50, an arm plate 51 connected to the base plate 50 at an angle, an infrared pass filter 52 mounted to the base plate 50, and a magnet 53 disposed on the arm plate 51 for connecting with the metal piece 24 by magnetic force to thereby attach the magnetic clip-on filter module 5 to the housing 20. The magnetic clip-on filter module 5 is so configured that when the magnet 53 is connected to the metal piece 24, the infrared pass filter 52 is aligned with the second lens 222. The infrared pass filter 52 is configured to permit passage of light having a wavelength that falls within a specific range of near infrared, and to block light having a wavelength that falls outside of the specific range of near infrared. In accordance with some embodiments, the specific range of near infrared corresponds to a spectrum of near infrared emitted by the NIR light source 4. For example, in a case where the NIR light source 4 is configured to emit infrared light with a center wavelength of 940 nm, the infrared pass filter 52 that has a specific range of about 940±20 nm may be used in the magnetic clip-on filter module 5; in a case where the NIR light source 4 is configured to emit infrared light with a center wavelength of 850 nm, the infrared pass filter 52 that has a specific range of about 850±20 nm may be used in the magnetic clip-on filter module 5. In a case where it is intended to fuse only the thermal image and the NIR image to obtain the assessment image, the use of the magnetic clip-on filter module 5 that is equipped with the infrared pass filter 52 can reduce interference from visible light, and the assessment image may thus be clearer and have finer details. In certain cases, the infrared pass filter 52 may be replaced by a visible light pass filter or an infrared cut filter, so the multi-spectral camera device 2 can obtain a fusion image that combines the thermal image and the VIS image to serve as the assessment image.

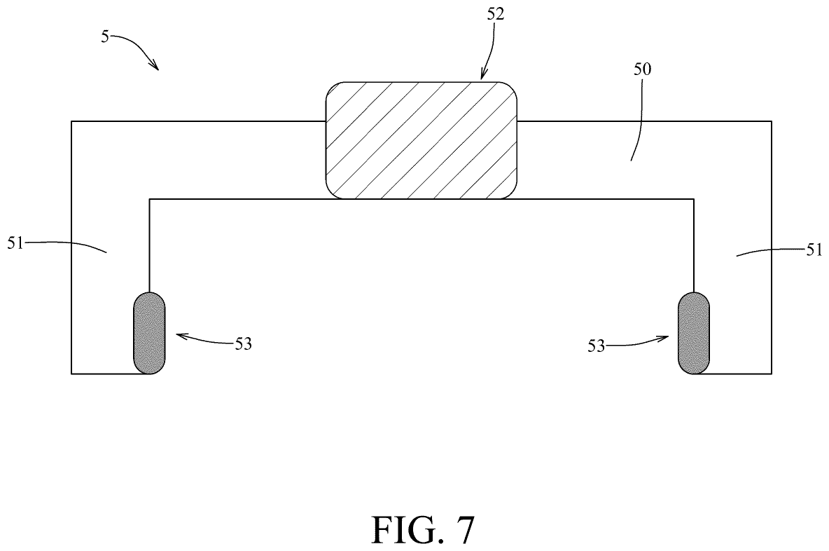

FIG. 7 illustrates a variation of the magnetic clip-on filter module 5. In this variation, the magnetic clip-on filter module 5 is U-shaped, and includes a pair of arm plates 51 that are connected to the base plate 50 at opposite sides, and a pair of magnets 53 that are attached to inner surfaces of the arm plates 51, respectively. In this scenario, the multi-spectral camera device 2 (see FIG. 4) may correspondingly include a pair of metal pieces 24 (only one is shown from the perspective of FIG. 4) attached respectively to opposite lateral surfaces of the housing 20 that are each connected to the front surface of the housing 20 at an angle. This configuration may result in a firmer connection between the housing 20 and the magnetic clip-on filter module 5.

The scenario of FIG. 1 is suitable for performing the quick screening method on people with limited mobility, such as a person sitting in a wheelchair. The person can position their feet onto the testing plate 100 by simply stepping on the testing box 1, while the multi-spectral camera device 2, with or without the use of the magnetic clip-on filter module 5, is placed in the accommodation space of the testing box 1 and under the testing plate 100, with the first lens 212 and the second lens 222 (see FIGS. 2 and 4) facing upward. The testing plate 100 receives heat from the feet, and thus reflects the temperature distribution of the feet. Then, the multi-spectral camera device 2 is operated through the computerized device 3 to simultaneously capture a thermal image and an NIR image in the direction of the soles of the feet through the testing plate 100, where the thermal image shows the temperature distribution of the testing plate 100 that reflects the temperature distribution of the feet, and the NIR image shows clear details and contours of the feet. It is noted that the opaque wall 11 may reduce interference from other electromagnetic waves, including visible light, on the multi-spectral camera device 2 and the image capturing process. Furthermore, the opaque wall 11 is configured to have a height that is sufficient for the multi-spectral camera device 2 to capture an image that includes the entire testing plate 100 when the multi-spectral camera device 2 is placed on the ground and covered by the testing box 1.

In accordance with some embodiments, the processor 23 performs image fusion using alpha blending. For example, the processor 23 sets the thermal image as a foreground image and the NIR image or VIS image as a background image, and then multiplies the foreground image by an alpha value α that ranges between 0 and 1 (i.e., from 0% to 100%), and multiplies the background image by (1−α). Subsequently, the processor 23 aligns the processed foreground image with the processed background image and overlays the two to form an alpha-blended image that serves as the assessment image. The processor 23 then transmits the assessment image to the computerized device 3 through a communication module (e.g., a Wi-Fi module, not shown).

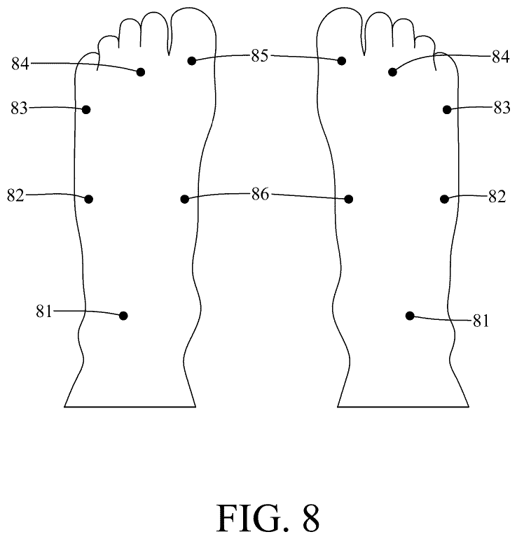

The computerized device 3 may display the assessment image on a screen. The assessment image may use a variety of colors and/or brightness levels to represent different temperatures within a temperature range. In accordance with some embodiments, the temperature range is set to 20° C. to 40° C. For some areas of the feet that require special attention, the temperature range may be narrowed to, for example, 32° C. to 39° C., so as to increase accuracy of recognition. In accordance with some embodiments, the software program executed by the computerized device 3 may show numerical temperatures on the screen for specific areas of the feet, such as an area with the highest temperature, an area with the lowest temperature, an average temperature of a selected area, an area of interest, etc. In accordance with some embodiments, the computerized device 3 determines, based on the assessment image, whether a temperature difference between a predetermined portion of one foot and a corresponding portion of the other foot (hereinafter referred to as predetermined corresponding portions of the feet) exceeds 2.2° C., and reports a potential case of diabetic foot syndrome when the temperature difference exceeds 2.2° C. by, for example, displaying a message on the screen. In accordance with some embodiments, the predetermined corresponding portions of the feet may be key areas where healthcare professionals would focus on in traditional tactile assessments for patients with diabetic foot syndrome, such as the portions 81 to 86 illustrated in FIG. 8. In accordance with some embodiments, the assessment image may be provided to a medical institution, such as a hospital or a clinic, to perform further analysis. In accordance with some embodiments, the assessment image may be used for remote consultation, so that the person can undergo preliminary medical diagnoses without needing to visit a medical institution, thereby reducing consumption of medical resources, alleviating workload of healthcare professionals, and decreasing frequency with which patients in rural areas need to travel to urban medical institutions. In accordance with some embodiments, the aforesaid determination and reporting may be performed by a medical professional. In accordance with some embodiments, the assessment image and the determination thus made may be stored in a database for future use, such as for training an artificial intelligence (AI) model for medical applications. In accordance with some embodiments, the multi-spectral camera device 2 transmits the thermal image, the NIR image and the VIS image to the computerized device 3, and the image fusion is performed by the computerized device 3 rather than by the multi-spectral camera device 2.

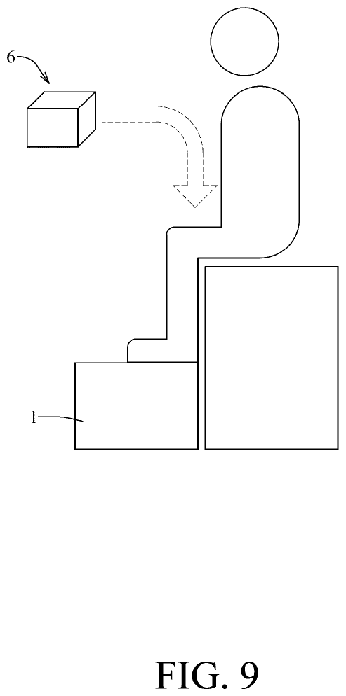

Referring to FIGS. 1 and 9, the system may be used in a pressure test. During the pressure test, the person undergoing the test is in a seated position with their thighs and calves forming an angle of about 90 degrees. Then, a heavy object 6 (e.g., weighing more than 10 kilograms or 20 kilograms) may be placed on the thighs of the person when the multi-spectral camera device 2 is capturing the thermal image and the NIR image for a pressure test. According to experiments, the pressure exerted on the feet is positively correlated with a plantar temperature displayed in the assessment image.

In accordance with some embodiments, the assessment image is further used for analysis of changes in the appearance of the feet over time. The computerized device 3 may compare the latest assessment image with another assessment image that was captured on an earlier day (i.e., that was captured one or multiple days ago), and determine whether a variation between the two images in terms of an area or a shape of one of the feet does not conform with a predetermined criterion. When the comparison result indicates that the variation does not conform with the predetermined criterion, the computerized device 3 may report a potential case of diabetic foot syndrome by, for example, displaying a message on the screen.

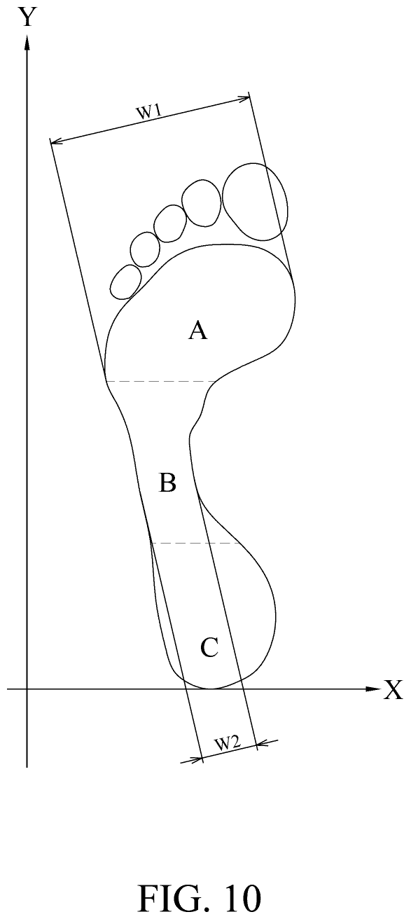

FIG. 10 illustrates an exemplary footprint in the assessment image. In accordance with some embodiments, an area or a shape of a foot can be evaluated by edge detection that detects a contour of the footprint in the assessment image. In accordance with some embodiments, an area or a shape of a foot can be evaluated by calculating a definite integral of a selected area of the footprint, such as areas A, B, and/or C in FIG. 10. By comparing the contours and/or sizes of the selected areas of the footprints captured on different days, the computerized device 3 or medical professionals may be able to determine whether the variation is normal (i.e., conforming with the predetermined criterion), and to assess a risk of diabetic foot syndrome accordingly. In accordance with some embodiments, the footprint in the assessment image can be used to obtain a foot progression angle (i.e., an angle between a centerline of the footprint and a direction of movement of the person), which can be used to assess whether there is swelling or atrophy on the sole of the foot. In accordance with some embodiments, the footprint in the assessment image can be used to obtain a width (W1) of the widest portion of the foot, and a width (W2) of the narrowest portion of the foot, which can also be used to assess whether there is any abnormality on the sole of the foot. These changes in appearance can be clearly observed from the NIR image.

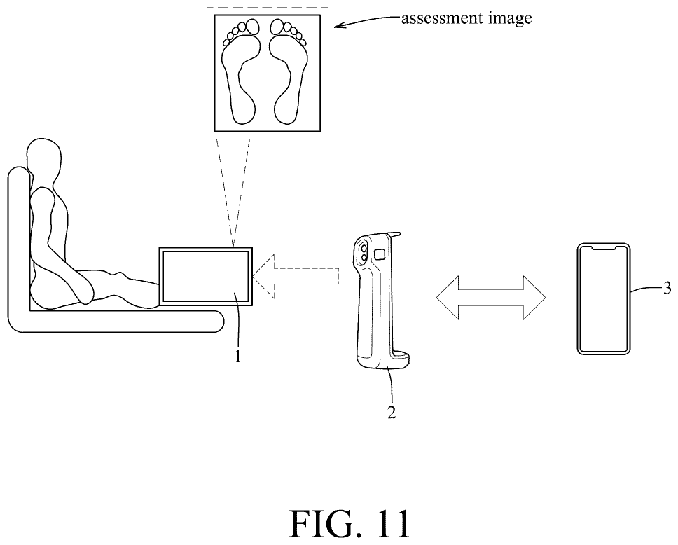

FIG. 11 illustrates another scenario of implementing the quick screening method in accordance with some embodiments. In this scenario, the person is seated, and the testing box 1 is positioned between the person and the multi-spectral camera device 2 at a height corresponding to the hip of the person, with an opening of the testing box 1 facing the person, so that the outer surface of the testing plate 100 faces the multi-spectral camera device 2, and the inner surface of the testing plate 100 faces the person. The person stretches out their legs, and places their feet into the accommodation space of the testing box 1, with the soles of the feet in contact with the inner surface of the testing plate 100. Then, the multi-spectral camera device 2 is able to obtain the assessment image through the testing plate 100.

In summary, the system of this disclosure uses the multi-spectral camera device 2 to obtain the assessment image that combines the thermal image and the NIR image of the soles of the feet, which can be used to not only compare the temperatures of corresponding areas of the feet, but also to observe the appearance of the feet, thereby assessing diabetic foot syndrome. The test box 1 facilitates acquisition of a clear footprint in the assessment image from the person, and enables the person to perform the quick screening in different positions. Compared to conventional methods for inspecting diabetic foot syndrome, using the system of this disclosure to perform the quick screening is relatively simple and time-efficient, which may motivate people to perform self-assessments periodically, and assist in early identification of symptoms of diabetic foot syndrome.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A quick screening method for assessing diabetic foot syndrome for a person, comprising:
positioning both feet of the person onto a testing plate that blocks visible light and permits passage of near infrared (NIR);
using a multi-spectral camera device to simultaneously capture a thermal image and an NIR image of soles of the feet of the person from a side of the testing plate that is opposite to the feet of the person;
performing image processing to align the thermal image with the NIR image and overlay the thermal image and the NIR image, thereby obtaining an assessment image of the feet;
determining, based on the assessment image, whether a temperature difference between a predetermined portion of one of the feet and a corresponding portion of the other one of the feet exceeds 2.2° C.; and
reporting a potential case of diabetic foot syndrome in response to determining that the temperature difference exceeds 2.2° C.

2. The quick screening method as claimed in claim 1, further comprising:
comparing the assessment image with another assessment image that was captured on an earlier day; and
reporting a potential case of diabetic foot syndrome in response to a comparison result indicating that a variation between the assessment image and the another assessment image in terms of one of an area and a shape of one of the feet does not conform with a predetermined criterion.

3. The quick screening method as claimed in claim 1, further comprising:
using an NIR light source to illuminate the feet of the person when the multi-spectral camera device is capturing the thermal image and the NIR image.

4. The quick screening method as claimed in claim 1, wherein the testing plate forms a central portion of a main wall of a testing box that includes an opaque wall connected to a periphery of the main wall, the main wall and the opaque wall cooperatively define an accommodation space of the testing box, and
wherein the positioning both feet of the person onto the testing plate includes:
positioning the testing box at a height corresponding to a hip of the person, with an outer surface of the testing plate facing the multi-spectral camera device, and an inner surface of the testing plate facing the person; and
placing the feet of the person into the accommodation space of the testing box, with the soles of the feet in contact with the inner surface of the testing plate.

5. The quick screening method as claimed in claim 1, wherein the testing plate forms a central portion of a main wall of a testing box that includes an opaque wall connected to a periphery of the main wall, and the main wall and the opaque wall cooperatively define an accommodation space of the testing box, and
wherein the positioning both feet of the person onto the testing plate includes:
placing the multi-spectral camera device on a ground, with a lens module of the multi-spectral camera device facing upward;
covering the multi-spectral camera device with the testing box, such that the multi-spectral camera device is disposed in the accommodation space of the testing box, an inner surface of the testing plate faces the lens module of the multi-spectral camera device, and an outer surface of the testing plate faces upward; and
placing the feet of the person onto the outer surface of the testing plate.

6. The quick screening method as claimed in claim 5, wherein the person is seated when the feet are placed onto the outer surface of the testing plate, and said quick screening method further comprises:
placing an object that weighs more than 10 kilograms over thighs of the person when the multi-spectral camera device is capturing the thermal image and the NIR image.

7. The quick screening method as claimed in claim 1, wherein the testing plate has dimension markings formed thereon.

8. The quick screening method as claimed in claim 1, wherein the multi-spectral camera device includes:
a first camera module including a first image sensor that is sensitive to far infrared for capturing the thermal image resulting from a temperature distribution on the testing plate, and a first lens that corresponds in position to the first image sensor, where the temperature distribution on the testing plate is related to temperature distribution of the feet of the person; and
a second camera module including a second image sensor that is sensitive to near infrared for capturing the NIR image, and a second lens that corresponds in position to the second image sensor.

9. The quick screening method as claimed in claim 8, wherein the multi-spectral camera device further includes:
a housing that accommodates the first image sensor and the second image sensor, and that has a front surface to which the first lens and the second lens are attached, and a lateral surface which is connected to the front surface;
a metal piece attached to the lateral surface of the housing; and
a magnetic clip-on filter module configured to be detachably mounted to the housing, and having a base plate, an infrared pass filter mounted to the base plate, an arm plate connected to the base plate, and a magnet disposed on the arm plate for connecting with the metal piece by magnetic force,
wherein the infrared pass filter is configured to permit passage of light having a wavelength that falls within a specific range of near infrared, and to block light having a wavelength that falls outside of the specific range of near infrared, and
wherein the magnetic clip-on filter module is configured in such a way that, when the magnet is connected to the metal piece, the infrared pass filter is aligned with the second lens.
